SEQUENCE LISTING (8 sequences)

SEQ ID NO: 1 - DNA, 27 bp, Artificial
  SK3-1B-specific primer
cctccatctccactccctctgggaggg

SEQ ID NO: 2 - DNA, 20 bp, artificial
  SK3-1B-specific primer
cccctcctccgtcttggggc

SEQ ID NO: 3 - DNA, 25 bp, artificial
  Forward primer designed to anneal to sequence unique to the distinct initial exon of the SK3 transcript
tgttatggtgatagagaccgagctc

SEQ ID NO: 4 - DNA, 18 bp, artificial
  Forward primer designed to anneal to sequence unique to the distinct initial exon of an SK3 transcript
agccccaagacggaggag

SEQ ID NO: 5 - DNA, 24 bp, artificial
  Reverse primer designed to anneal to sequences in the shared exon 2 of SK3-1B.
tggacagactgataaggcatttca

SEQ ID NO: 6 - DNA, 20 bp, artificial
  Reverse primer designed to anneal to sequences in the shared exon 2 of SK3-1B.
ggccaacgaaaacatggagt

SEQ ID NO: 7 - DNA, 31 bp, artificial
  SK3-1B-specific primer
tgtactcaaaggactccatgttttcgttggc

SEQ ID NO: 8 - DNA, 25 bp, artificial
  SK3-1B-specific primer
tcccagagggagtggagatggagga

(19) United States
(12) Patent Application Publication
(10) Pub. No.: US 2005/0010967 A1
(43) Pub. Date: Jan. 13, 2005

(54) SK3-1B GFP transgenic mouse model for spinocerebellar ataxia and hyperexcitable behavior

(75) Inventors: Vikram Shakkottai, St. Louis, MO (US); K. George Chandy, Laguna Beach, CA (US); Frank LaFerla, Irvine, CA (US)

Correspondence Address: CHRISTIE, PARKER & HALE, LLP, PO BOX 7068, PASADENA, CA, 91109-7068, US

(73) Assignee: The Regents of the University of California

(21) Appl. No.: 10/791,316

(22) Filed: Mar. 1, 2004

Related U.S. Application Data
(60) Provisional application No. 60/451,381, filed on Feb. 28, 2003.

(51) Int. Cl. A01K 67/027

(52) U.S. Cl. 800/18

(57) ABSTRACT

Transgenic animals exhibiting several hallmark features of SK-3 related pathologies, including moderate to severe ataxia, intention tremors and hyperexcitable behavior are provided. This transgenic animal model represents a valuable research tool for testing candidate agents for treatment of individuals diagnosed with SK-3 related pathologies, either prophylactically or after disease onset.

### CROSS-REFERENCE TO RELATED APPLICATION(S)

This application claims the benefit of U.S. Provisional Application, Ser. No. 60/451,381, filed Feb. 28, 2003, the contents of which is hereby incorporated by reference in its entirety.

### STATEMENT REGARDING FEDERALLY SPONSORED RESEARCH OR DEVELOPMENT

This invention was made with Government support under Grant No. NIH MH59222, awarded by the National Institutes of Health. The Government may have certain rights in this invention.

### INTRODUCTION

A neuronal action potential is followed by an afterhyperpolarization current (IAHP) which regulates spike frequency. Fast, medium and slow components of IAHP are distinguished, the fast component being mediated by large-conductance calcium-activated K⁺ channels, while small-conductance calcium-activated K⁺ (SK) channels are thought to be responsible for the medium, and possibly the slow components of this current. Functional SK channels are formed from the homo- or heterotetrameric association of products of three related genes, SK1-SK3 (also known as KCNN1-KCNN3 and SKCa1-SKCa3).

Several lines of evidence have implicated the SK3 channel in schizophrenia, and more recently in anorexia nervosa and ataxia, although the results are not conclusive. The SK3 gene is located on human chromosome 1q216 in a region containing a major susceptibility locus for familial schizophrenia and familial hemiplegic migraine associated with permanent cerebellar ataxia. SK3 is expressed abundantly in the regions implicated in schizophrenia including the hippocampus, the limbic system and midbrain regions rich in monoaminergic neurons. The SK3 channel functions as the intrinsic pacemaker in rat dopaminergic neurons, and specific pharmacological blockade of SK channels abolishes the medium IAHP leading to bursting action potentials and increased dopamine release, consistent with the dopamine model of schizophrenia. SK3 contains two polymorphic polyglutamine repeats in its N-terminus. Multiple association studies find overrepresentation of longer alleles of the second SK3 polyglutamine repeat in schizophrenia patients, although other such studies have failed to confirm this association. SK3-D, a rare truncation mutant of SK3 identified in a patient with schizophrenia, potently suppressed the entire family of endogenous SK currents in mammalian cells in a dominant negative fashion. Such dominant-inhibitory behavior in dopaminergic pathways of the nervous system should have functional consequences similar to pharmacological blockade of SK channels: bursting action potentials and increased dopamine release. Under the dopamine model of schizophrenia, such changes would predispose to the development of disease.

The SK3 gene and protein are described in U.S. Pat. No. 6,165,719, the entire contents of which are incorporated by reference.

### SUMMARY OF THE INVENTION

The present inventors have isolated a novel SK3 splice variant, SK3-1 B, which encodes a truncated product that suppresses SK channels in a dominant-negative manner. Such molecular suppression of endogenous SK channels in the brain should enhance neural excitability and induce calcium mediated excitotoxicty, analogous to the effect of the SK channel blocking neurotoxin apamin. To test this idea, the present inventors generated transgenic mice over-expressing SK3-1B in the brain under control of Thy1.2-SX, a neuron-specific promoter. Eleven transgenic founders have been identified and of these seven exhibit progressively worsening ataxia, intention tremor, and hyper-excitable behavior. The symptoms started at the 7th-8th week of life and progressively worsened.

These transgenic mice and all future Tg mice over-expressing the SK3-1B under different promoters, may serve as excellent models for neuropsychiatric diseases including hereditary ataxias, schizophrenia, anorexia nervosa and attention deficit disorder. Such models could be used to dissect pathogenic mechanisms underlying these diseases, in evaluating potential therapeutic agents, and in the development of new drugs for these diseases.

Accordingly, one embodiment of the present invention provides a transgenic mouse whose genome comprises a transgene encoding human small conductance calcium-activated potassium (SK) channel protein, splice variant B1 (“SK3-1B”), wherein the transgene is operably linked to a neuron-specific promoter, and wherein expression of the transgene results in ataxia.

Another embodiment of the present invention provides a transgenic mouse whose genome comprises a transgene encoding SK3-1B, wherein the transgene is operably linked to a neuron-specific promoter, and wherein expression of the transgene results in an intention tremor.

Another embodiment of the present invention provides a transgenic mouse whose genome comprises a transgene encoding SK3-1B, wherein the transgene is operably linked to a neuron-specific promoter, and wherein expression of the transgene results in hyperexcitable behavior.

Another embodiment of the present invention provides a method of screening biologically active agents that facilitate reduction of ataxia in vivo, the method comprising administering a candidate agent to a transgenic mouse whose genome comprises a transgene encoding SK3-1B, and determining the effect of said agent upon the level of ataxia.

Another embodiment of the present invention provides a method of screening biologically active agents that facilitate reduction of intention tremors in vivo, the method comprising administering a candidate agent to a transgenic mouse whose genome comprises a transgene encoding SK3-1B, and determining the effect of said agent upon the level of intention tremors.

Another embodiment of the present invention provides a method of screening biologically active agents that facilitate improvement in hyperexcitable behavior, the method comprising administering a candidate agent to a transgenic mouse whose genome comprises a transgene encoding SK3-1B, and determining the effect of said agent upon hyperexcitable behavior.

### BRIEF DESCRIPTION OF THE DRAWINGS

FIG. 1. Genomic organization, splice junctions and putative protein products of SK3 and SK3-1B. (a) Exon-intron organization of the SK3 locus showing the location of exon 1B. The SK3 and SK3-1B cDNAs are shown (not to scale). (b) 5′ and 3′ boundaries of the introns that lie between exons 1, 1B and 2. The AUG shown in the SK3-1B protein was determined by the NetStart1.0 (www.cbs.dtu.dk/services/Netstart) program (score 0.590; score >0.5 being significant) to be a reasonable translation initiation codon. There are no in-frame potential translation start sites upstream. An in-frame AUG that corresponds more closely to the Kozak consensus sequence (score 0.752) lies 132 bp downstream to the AUG shown, and if utilized would produce an ORF of 1122 bp. (c) Putative protein product of SK3 showing transmembrane segments, the calmodulin (CAM)-binding site in the C-terminus, and the polyglutamine repeats in the N-terminus. An arrow indicates the presumed start site of SK3-1B.

FIG. 2. Distribution of SK3 and SK3-1B mRNA in human tissues determined by TAQMAN™ quantitative RT-PCR. (a-c) Pooled human tissues. (d-f) brain regions from a single donor: SK3 (a, d) and SK3-1B (b, e) mRNA copy number per microliter cDNA expressed as mean 7SD. (c, f) SK3-1B/SK3 ratio displayed as a percentage. The horizontal lines in (a) and (b) show the arbitrary division into abundant, intermediate and low expression levels. *No detectable transcript.

FIG. 3. Dominant negative suppression of endogenous SK currents in PC12 cells by SK3-1B. SK and Kᵥ currents in untransfected PC12 cells. Activation of SK current in PC12 with 1 μM Ca²⁺ in the internal solution; Kᵥ currents are visible at depolarized potentials. (a) SK but not Kᵥ current is blocked by 100 nM apamin. (b) In the absence of internal Ca²⁺ in the pipette solution, SK currents are not visible but Kᵥ currents remain unchanged. SK3-1B-GFP (c) and SK3-1B-IRES-GFP (d) suppress SK but not Kᵥ currents. GFP- (e) and GFP-Kv1.7 (f) do not affect SK and KV currents. All SK recordings were done with symmetric (160 mM) K⁺ as internal and external recording solutions. KV current amplitude was determined with an external solution containing 160 mM Na⁺ aspartate.

FIG. 4. Scatter plot showing the effect of SK3-1B-GFP and SK3-1B-IRES-GFP on endogenous SK and Kᵥ currents in PC12 cells. SK currents (mean±SEM) in untransfected (open squares; 6.24±1.57 nS, n=22), SK3-1B-GFP-transfected (open triangles, 0.68±0.08 nS, n=11; p=0.018) and SK3-1B-IRES-GFP-transfected PC12 cells (open circles; 1.1±0.03 nS, n=12; p=0.024). Kᵥ currents (mean±SEM) in untransfected (filled squares; 707.94±147.7 pA, n=15), SK3-1B-GFP-transfected (filled triangles; 475.96±61.78 pA, n=15; p>0.05) and SK3-1B-IRES-GFP-transfected PC12 cells (filled circles; 560.37±75.9 pA, n=14; p>0.05). The data were analyzed by a Student's t-test between the respective transfected cell population and the untransfected PC12 cells.

FIG. 5. Dominant negative suppression of endogenous SK2 currents in Jurkat T lymphocytes by SK3-1B. (a) SK2 and Kv1.3 currents in untransfected Jurkat T lymphocytes; SK2 but not Kv1.3 is blocked by 100 nM Lei-Dab. (b) SK2 and Kv1.3 currents in SK3-1B-GFP-transfected Jurkat T lymphocytes.; no SK2 current is observed (n=4), while the Kv1.3 current resembles that in untransfected cells. (c) SK2 and Kv1.3 currents in GFP-transfected Jurkat T lymphocytes (n=4). All experiments were done with symmetric (160 mM) K⁺ as internal and external recording solutions.

FIG. 6. Altered sub-cellular expression pattern of native SK3 in PC12 cells transfected with SK3-1B. (a) Untransfected PC12 cells immunolabeled with anti-SK3 antibody; Nomarski image (upper panel), annular SK3 staining pattern (middle panel), intensity histogram of SK3 fluorescence (lower panel). Pre-incubation with blocking peptide abolished staining confirming the specificity of the antibody (data not shown). (b) SK3-1B-GFP-transfected cells stained for SK3; SK3-1B-GFP (upper panel), intracellular speckled SK3 staining (middle panel), intensity histogram of SK3 fluorescence (lower panel). The anti-SK3 antibody does not react with SK3-1B, which lacks the SK3 N-terminus (data not shown). (c) GFP-vector transfected cell; GFP (upper panel), annular SK3 staining (middle panel), and intensity histogram of SK3 fluorescence resembles that of untransfected cells (lower panel). (d). GFP-Kv1.7-transfected cell: GFP-Kv1.7 (upper panel), annular SK3 staining (middle panel) and normal intensity histogram (lower panel). The average intensity histograms shown are based on data derived from six cells.

FIG. 7. Southern blots confirming genotype. Genotype analysis for F1s of founder line D11 (Left) and D2 (Right). The major 6.4 kb Tg band is shown by an arrow along with other Tg bands. A mixture of probes was used. Representative intensity histograms for the Tg are shown below. The area under each histogram used to calculate the copy number is shown below each histogram. Copy number was calculated as (area under Tg band/½×area under endogenous band).

FIG. 8. Expression profile of SK3-1B-GFP in transgenic lines. Expression in deep cerebellar neurons (A). Magnified view in (B). Abundant expression in Layer V of the neocortex in D11 founder line (C). Expression in spinal cord (D) and magnified view of expression in anterior horn neurons (E). Non-transgenic littermate shows no staining (F).

FIG. 9. Haematoxylin and Eosin staining of non-Tg (Left) and Tg cerebellum (Right).

FIG. 10. GFAP immunostaining of indicated areas in the two Tg founder lines D11 (Left), D2 (Middle) and Non-Tg animals (Right).

FIG. 11. Rotorod analysis at 8 weeks for D11 (Left) and D2 (Right). In each case non-Tg shown in black and Tg shown in red. Each point represents the mean of six trials and the error bars represent the SEM.

FIG. 12. Latencies on the non-rotated rod (Left) and hanging wire (Right) for the D11 and D2 transgenic lines. Bars show mean latencies with error bars indicating the SEM.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

The term “transgene” refers to the genetic material which has been or is about to be artificially inserted into the genome of an animal, particularly a mammal and more particularly a mammalian cell of a living animal.

“Transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

“Operably linked” means that a DNA sequence and a regulatory sequence(s) are connected in such a way as to permit gene expression when the appropriate transcriptional activator proteins are bound to the regulatory sequence(s). For example, a nucleic acid sequence encoding SK3-1B may be operably linked to a neuron-specific promoter to facilitate production of SK3-1B polypeptide in brain cells.

“Neuron-specific promoter” refers to a regulatory sequence(s) operably linked to an SK3-1B-encoding nucleic acid in such a way as to permit gene expression of the SK3-1B-encoding nucleic acid in neurons with little or no expression of the SK3-1B-encoding nucleic acid in non-neurons. One such neuron-specific promoter is the Thy1.2-SX promoter; other examples are known to those of skill in the art.

“SK3-related pathologies” refers to a set of neuropsychiatric diseases associated with SK-3, including hereditary ataxias, schizophrenia, anorexia nervosa and attention deficit disorder.

### Overview of the Invention

The present inventors have discovered a novel alternative transcript of the SK3 (a.k.a. SKCa3/KCNN3) potassium channel that utilizes a novel exon 1 that splices to the remaining normal SK3 exons. This novel SK3 transcript, SK3-1B, produces a truncated protein that dominant negatively suppresses functional SK3 and SK2 currents. We expressed SK3-1B in a transgenic (Tg) mouse under a neuron specific promoter. The Tg mouse develops ataxia, intention tremor, and gait incoordination suggestive of a cerebellar ataxia. Immunoperoxidase staining for the transgene revealed expression in the deep cerebellar neurons (DCN), layer V of the neocortex, brain stem nuclei, subiculum, CA1 of the hippocampus, and anterior horn neurons in the spinal cord. The expression profile, especially in the deep cerebellar neurons is consistent with the ataxic character of the phenotype. Electrophysiological analysis in the Tg DCN suggests that there is a loss of normal pacemaker firing in these neurons with short intervals of burst firing. There does not appear to be complete suppression of the SK currents in these neurons suggesting that an SK channel opener may reverse the phenotype. Suppression of SK channels in other areas of the CNS where SK channels are postulated to be important, such as the midbrain dopaminergic neurons, may induce abnormal firing in these neurons with increased dopamine release. Expression of SK3-1B in dopaminergic neurons under a tyrosine hydroxylase promoter may generate a mouse model of dopamine excess with correlates to human schizophrenia.

The animals of the present invention are genetically altered so as to overexpress human SK3-1B in a tissue-specific manner. Specifically, expression of SK3-1B is targeted to the brain using neuron-specific promoters, such as the Thy1.2-SX promoter. The transgenic animals may be either homozygous or hemizygous for the genetic alteration.

The transgenic animals of the present invention exhibit several hallmark features of SK-3 related pathologies, including moderate to severe ataxia, intention tremors and hyperexcitable behavior. Thus, this transgenic animal model represents a valuable research tool for testing candidate agents for treatment of individuals diagnosed with SK-3 related pathologies, either prophylactically or after disease onset.

### Truncated SK3 Isoform

5′ RACE and determination of genomic organization. The sequence of the SK3-1B cDNA was extended by 5′-RACE (Rapid Amplification of cDNA Ends) with an AP1 primer (Clontech, Palo Alto, Calif., USA) and an SK3-1B-specific primer (5′-CCTCCATCTCCACTCCCTCTGGGAGGG-3′), using human adult MARATHONREADY™ cDNA (Clontech) as the template. Subsequently, a nested PCR was carried out using the RACE product and an SK3-1B-specific primer (5′-CCCCTCCTCCGTCTTGGGGC-3′) and an AP2 primer (Clontech). The longest product (338 bp) was purified using a QIAQUICK™ gel extraction kit, ligated into PCR 2.1 vector and sequenced.

Preparation of total RNA and real-time quantitative RT-PCR. Human total RNA master panel (Clontech) was used to profile the expression pattern of SK3-1B. Total RNA was isolated from nine brain regions of human adult brain tissue using TRIZOL reagent (Life Technologies, Inc.) as per the manufacturer's protocol. Total RNA (1 μg) was used as a template for first-strand cDNA synthesis using poly-T primers (TAQMAN™ reverse transcription reagents, Applied Biosystems). The mRNA for each SK3 transcript was measured by real-time quantitative RT-PCR using a Prism model 7700-sequence detection instrument (PE Applied Biosystems). Forward and reverse primers, and TAQMAN™ fluorescent probes were designed by Primer Express version 1.5 (Applied Biosystems). Forward primers were designed to anneal to sequences unique to the distinct initial exons of the two SK3 transcripts. The sequences of forward primers were 5′-TGTTATGGTGATAGAGACCGAGCTC-3′ for SK3 and 5′-AGCCCCAAGACGGAGGAG-3′ for SK3-1B. The reverse primers, designed to anneal to sequences in the shared exon 2, were 5′-TGGACAGACTGATAAGGCATTTCA-3′ for SK3 and 5′-GGCCAACGAAAACATGGAGT-3′ for SK3-1B. The TAQMAN™ fluorescent probes (5′-labeled with 6FAM, and 3′-labeled with TAMRA as a quencher), designed to anneal to sequences between the forward and reverse primers, were 5′-TGTACTCAAAGGACTCCATGTTTTCGTTGGC-3′ for SK3 and 5′-TCCCAGAGGGAGTGGAGATGGAGGA-3′ for SK3-1B. The SK3 and SK3-1B amplification products were 92 and 76 bp, respectively. The threshold cycle, Cₜ, which correlates inversely with the target mRNA levels, was measured as the cycle number at which the reporter fluorescent emission increased above a pre-set threshold level. To obtain absolute quantification, standard curves were plotted for every assay and were generated using defined concentrations of SK3-1B in Image clone 4139388, and SK3 cDNA cloned in pcDNA3.1 HisB. Standard curves for each amplicon were plotted from eight different concentrations of standards, each run in triplicate. Concentrations were determined by spectrophotometry and purity confirmed by agarose gel electrophoresis. Purified clones were diluted to eight different concentrations and stored in single-use aliquots at −20° C., and the same diluted preparations were used throughout.

GFP constructs. Expressible constructs of the SK3 channel isoforms were produced with C-terminal GFP fluorescent tags to allow facile identification of expressing cells for electrophysiological recording. By inserting in-frame the SK3-1B coding region upstream to GFP in the pEGFP-N1 expression vector (Clontech), we generated SK3-1B-GFP. We also generated an SK3-1B construct containing GFP driven by an internal ribosome entry site (SK3-1B-IRES-GFP) by inserting the SK3-1B coding region into the pIRES2-EGFP expression vector (Clontech). The generation of the GFP-tagged Kv1.7 construct and the electrophysiological characterization of this tagged channel have been previously described. (Bardien-Kruger S, Wulff H, Arieff Z, Brink P, Chandy K G, Corfield V. Characterization of the human voltage-gated potassium channel gene, KCNA7, a candidate gene for inherited cardiac disorders, and its exclusion as cause of progressive familial heart block I (PFHBI). Eur J Hum Genet 2002; 10: 36-43.)

Cell culture. PC12 cells and Jurkat T lymphocytes were obtained from ATCC (Manassas, Va., USA). PC12 cells were cultured in Dulbecco's modified Eagle's medium containing 10% fetal calf serum and 2 mM glutamine in a 37° C. humidified incubator with 5% CO₂ and split 1:10 twice weekly. Jurkat T lymphocytes were cultured in RPMI containing 10% fetal calf serum, 2 mM glutamine and RPMI vitamins. Cells were split 1:4 every 2 days. Unless otherwise specified, all reagents were obtained from Sigma.

Transfection of constructs into PC12 and Jurkat cells. PC12 cells were plated in 12-well plates (5×105 cells/chamber) for 12-24 h, and then transiently transfected using FUGENE™ 6 (Roche Molecular Biochemicals, Indianapolis, Ind., USA) with the DNA construct of interest in serum-free OptiMEM medium (Life Technologies, Inc.) as per the manufacturer's recommended protocol. Cells were plated 36 h following transfection for overnight growth on glass coverslips and used for electrophysiological, immunolabeling and confocal microscopy experiments 48 h post-transfection. Jurkat T lymphocytes were transiently transfected using Xtremegene-Q2 transfection reagent (Roche) as per the manufacturer's recommended protocol. Cells were used for electrophysiological analysis 48 h following transfection.

Immunostaining. Following washing with Dulbecco's phosphate buffered saline (DPBS), cells were fixed with 4% paraformaldehyde for 20 min and washed 2× with DPBS. Permeabilization and blocking was done with 0.2% Triton-X-100 in 10% normal goat serum, followed by a wash with DPBS. Permeabilized cells were incubated with rabbit anti-SK3 antibody (Alomone Labs, Jerusalem, Israel) in a carrier solution containing 1% normal goat serum and 0.2 % sodium azide for 4 h at 4° C. Cells were subsequently washed and incubated with 1 μg/ml Alexa-Fluor 594 (referred to as Alexa-red)-conjugated goat-anti-rabbit-IgG (Molecular Probes) for 1 h at room temperature. The secondary antibody alone showed no signal above background (data not shown). SK3 antibody specificity was confirmed by preincubation of the primary antibody for 1 h with the blocking peptide supplied by the manufacturer (data not shown). As an additional test of antibody specificity, the anti-SK3 antibody was shown not to react with SK3-1B protein, an isoform that lacks the SK3 N-terminal epitope detected by the antibody (data not shown). Cells were mounted in 50% glycerol and stored in the dark prior to microscopic analysis.

Confocal microscopy. Images were collected with an MRC-1024 laser scanning confocal microscope (Bio-Rad, Hercules, Calif., USA) on an inverted Nikon Diaphot 200 stand using a 100× oil-immersion objective (Nikon, Melville, N.Y., USA). Confocal sections through the cell were taken by 1 mM increments of the focus motor. Laser power was maintained at 30% for image acquisition. Excitation wavelengths of 488 and 568 nM were used, and image collection was with 522/35 and 605/32 emission filters. Images were processed with Confocal ASSISTANT™ and ADOBE™ Photoshop 5.0.2. The Scion image 4.0.2 program (Scion Corporation, Frederick, Md., USA) was used to determine the pixel intensity of fluorescence that tagged presumptive intracellular and membrane SK3 protein. Three axes were drawn through each stained cell and pixel intensities were measured along each of these axes. The cell margins were defined as a rise of pixel intensity to 50% above background. The length of the axes between the cell margins was normalized to obtain the average intensity profile for each cell. The mean of the scaled intensity histograms was obtained for six native PC12 cells and six cells transfected with each GFP construct. The average ratio of presumptive membrane to intracellular fluorescence was estimated from these intensity histograms. The expression level of each transfected construct was quantitated as mean pixel intensity.

Electrophysiology. PC12 and Jurkat T cells were studied in the whole cell configuration of the patch clamp technique with a holding potential of −80 mV. We determined SK and Kᵥ current amplitude in untransfected cells or in cells transfected with SK3-1B-GFP, SK3-1BIRES-GFP or control constructs (GFP vector alone, GFP-Kv1.7). We used strategies previously employed for other types of K⁺ channels (Miller M J, Rauer H, Tomita H, Rauer H, Gargus J J, Gutman G A et al. Nuclear localization and dominant-negative suppression by a mutant SKCa3 N-terminal channel fragment identified in a patient with schizophrenia. J Biol Chem 2001; 276: 27 753-27 756; Tu L, Santarelli V, Sheng Z, Skach W, Pain D, Deutsch C. Voltagegated K⁺ channels contain multiple intersubunit association sites. J Biol Chem 1996; 271:18 904-18 911; Joiner W J, Khanna R, Schlichter L C, Kaczmarek L K. Calmodulin regulates assembly and trafficking of SK4/IK1 Ca²⁺-activated K⁺ channels. J Biol Chem 2001; 276: 37 980-37 985; Fanger C M, Rauer H, Neben A L, Miller M J, Rauer H, Wulff H et al. Calcium-activated potassium channels sustain calcium signaling in T lymphocytes. J Biol Chem 2001; 276: 12 249-12 256; Deutsch C. Potassium channel ontogeny. Annu Rev Physiol 2002; 64: 19-46) to determine whether SK3-1B could selectively suppress endogenous SK currents without affecting Kᵥ currents. This approach relies on the tetrameric structure of K⁺ channels and on the ability of SK subunits to coassemble specifically with other SK subunits. We overexpressed SK3-1B in PC12 and Jurkat cells to ensure that the majority of SK tetramers would contain at least one SK3-1B subunit. Brightly GFP-positive cells were identified under a fluorescence microscope to allow the analysis of cells expressing high levels of the channel constructs for electrophysiological studies. Control constructs were expressed at equivalently high levels as assessed by the intensity of the GFP signal.

To activate SK channels, the pipette solution contained (in mM): 145 K⁺ aspartate, 2 MgCl₂, 10 HEPES, 10 K₂EGTA, and 8.5 CaCl₂ (1 μM free Ca²⁺), pH 7.2, 290-310 mOsm. The external solution contained (in mM): 160 K⁺ aspartate (aspartate was used to minimize contributions of Cl⁻ current), 4.5 KCl, 2 CaCl₂, 1 MgCl₂, and 5 HEPES, pH 7.4, 290-310 mOsm. SK currents were elicited by 200-ms voltage ramps from −120 to 40 mV applied every 10 s, and slope conductance at −80 mV was taken as a measure of the SK conductance. Block of the SK current with apamin (BACHEM Bioscience Inc., King of Prussia, Pa., USA) or with Lei-Dab7 (gift from Jean-Marc Sabatier, University of Marseille) was observed as a reduction in slope conductance at −80 mV. To demonstrate the Ca²⁺ dependence of this current, we used an internal solution containing calcium-free KF. The amplitude of the K_{V} current was measured as the maximum current at 30 mV in 160 Na⁺ aspartate. Results obtained from untransfected and transfected cells were compared using Student's t-test.

SK3-1B lacks the N-terminus and S1 transmembrane segment. A BLAST search of GenBank with the human SK3 AJ251016 cDNA sequence identified a novel human SK3 EST (accession no. BF306047) derived from rhabdomyosarcoma cells. We sequenced the entire 1406 bp insert in the IMAGE clone no. 4139388 corresponding to this EST and found it to represent a novel transcript, henceforth referred to as SK3-1B. Using 5′ RACE on human brain mRNA with an SK3-1B-specific primer, we amplified and sequenced a 338 bp product and determined the approximate transcription start site of SK3-1B. The composite 1658 bp SK3-1B cDNA sequence has been deposited in GenBank (accession no. AY138900) and contains 377 bp of 5′ noncoding sequence and a 1254 bp open reading frame.

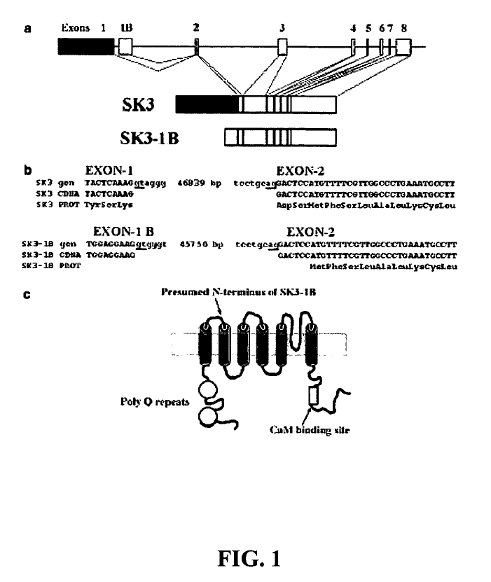

Comparison of the SK3-1B cDNA sequence with published genomic sequences (Sun G, Tomita H, Shakkottai V G, Gargus J J. Genomic organization and promoter analysis of human KCNN3 gene. J Hum Genet 2001; 46: 463-470; Accession nos: AF336797, AC034149, AC027645, AC025385) revealed the intron-exon organization of SK3-1B (FIG. 1a). SK3 and SK3-1B both utilize exons 2-8 and differ only at the 5′ end (FIG. 1a). Whereas SK3 reads through the entire first exon (exon 1A) and then splices into exon 2, 53 SK3-1B utilizes a novel exon (exon 1B) located 712 bp downstream from exon 1A, which splices to exon 2 (FIG. 1a, b). Exon 1B sequence has been deposited in GenBank and assigned accession no. AY138901. The splice donor and acceptor sites at the boundaries of the intervening sequences between exons 1, 1B and 2 are shown in FIG. 1b. Exon 1A encodes the 5′ noncoding sequence, the entire N-terminus and the S1 transmembrane domain of SK3, while exon-1B encodes only the 5′ noncoding sequence of SK3-1B; the initiation codon for the SK3-1B isoform is contained in exon 2.

The putative protein products of SK3 and SK3-1B are shown in FIG 1c. The SK3 protein (736 residues) is made up of a long N-terminus containing two polymorphic polyglutamine repeats, six transmembrane segments (S1-S6) and a C-terminus tightly complexed to calmodulin. In contrast, the putative SK3-1B protein (418 residues) begins in the same reading frame as SK3, but at the first methionine residue encoded by exon 2, eight residues upstream of the S2 transmembrane segment. It therefore lacks SK3's N-terminus and S1 segment (FIG. 1b).

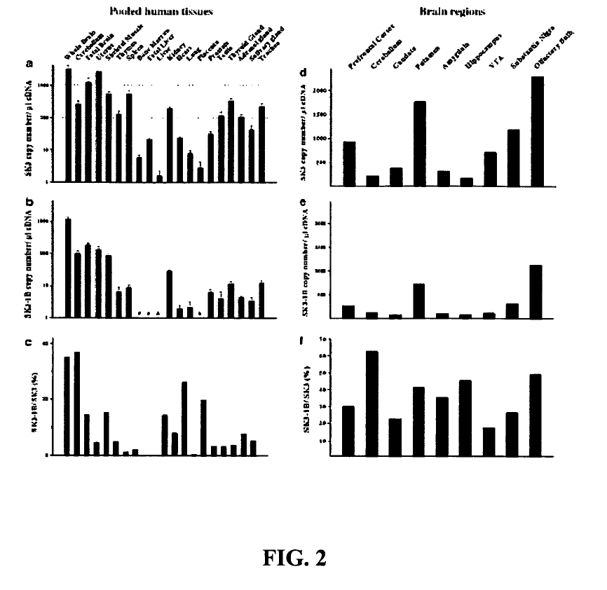

SK3-1B is widely expressed in the human brain. TAQMAN™ quantitative RT-PCR was used to determine the abundance of SK3 and SK3-1B transcripts in total RNA derived from tissues pooled from multiple human donors (Clontech). Each cDNA sample was analyzed 3-9 times and the mean copy number (7SD) per microliter of cDNA determined (FIG. 2). We have arbitrarily divided the expression levels into three groups (defined by horizontal lines in FIG. 2a, b), abundant (>1000 copies/μl cDNA), intermediate (100-1000 copies/μl cDNA) and low (<100 copies/μl cDNA). SK3 is expressed abundantly in adult and fetal brain and uterus, at intermediate levels in skeletal muscle, spleen, thymus, adrenal gland, thyroid, kidney, testis, trachea, and at low levels in bone marrow, fetal and adult liver, heart, lung, placenta, salivary gland and prostate (FIG. 2a). SK3-1B is present abundantly in the adult brain, at intermediate levels in fetal brain, cerebellum and uterus, and at lower levels in all the other tissues studied (FIG. 2b). The ratio of SK3-1B/SK3, displayed as a percentage, is shown in FIG. 2C. In the brain SK3-1B is present at between 15 and 60% of the level of SK3, and at significantly lower levels in other tissues.

TAQMAN™ RT-PCR was also performed on SK3 and SK3-1B products amplified from total RNA isolated from different brain regions of a single human donor (FIGS. 2d-f). SK3 was highly expressed in the olfactory bulb, putamen, prefrontal cortex, and in dopaminergic neurons in the ventral tegmental area and substantia nigra, and at lower levels in the caudate, amygdala, hippocampus and cerebellum (FIG. 2d), findings consistent with published expression data. SK3-1B was present in all these regions although at lower levels than SK3 (FIG. 2e). The ratio of SK3-1B/SK3 in the different brain areas of this donor varied from 15 to 60% (FIG. 2f). The high ratio of SK3-1B/SK3 in the pooled brain sample is consistent with the overall levels found in the single donor brain regions (cf FIG. 2c with f).

SK3-1B-GFP selectively suppresses endogenous SK currents in PC12 cells in a dominant-negative manner. GFP-tagged SK3 produces calcium-activated apamin-sensitive potassium currents when expressed in COS-7 cells, whereas SK3-1B-GFP exhibited no channel function when expressed alone (data not shown). Since the SK3-Δ truncation mutant and an N-terminal SK2 fragment were both reported to suppress SK currents in mammalian cell lines, presumably by coassembling with endogenous SK subunits into nonfunctional tetramers, the present inventors tested whether SK3-1B could exhibit similar dominant inhibitory activity. In support of this idea, protein fragments of Kᵥ and IK K⁺ channels corresponding to the region contained in SK3-1B have been shown to suppress Kᵥ and IK currents. We chose the rat pheochromocytoma cell line, PC12, as our experimental system since these cells natively express SK3 currents, as well as Kᵥ currents that would serve as an internal control. Two SK3-1B constructs were used: SK3-1B-GFP and SK3-1B-IRES-GFP. As controls, the inventors expressed GFP alone or an unrelated GFP tagged channel (GFP-Kv1.7). The expression level of each construct was quantitated as mean pixel intensity. SK3-1B-GFP was expressed at a slightly lower level (mean pixel intensity 108) than GFP-Kv1.7 (mean pixel intensity 180) or GFP vector alone (mean pixel intensity 174).

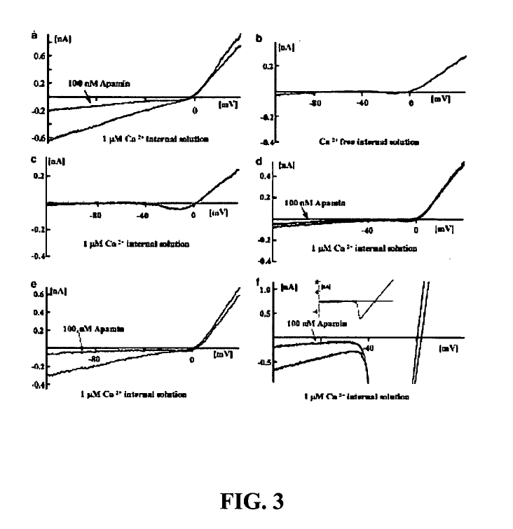

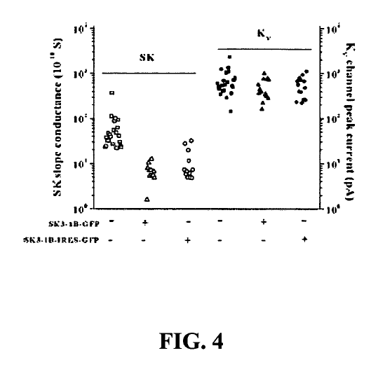

FIG. 3a demonstrates the endogenous SK3 and Kᵥ currents in PC12 cells. Current traces were elicited by voltage ramps from −80 to 40 mV. At potentials more negative than −40 mV, an inward SK3 current was seen if the pipette contained 1 μM free calcium (FIG. 3a), but was absent when calcium was omitted from the pipette solution (FIG. 3b). The calcium activated inward SK3 current was selectively blocked by apamin (100 nM), a potent peptide blocker of SK channels (FIG. 3a). The outward Kᵥ current observed at potentials more positive than 0 mV was unaffected by the internal calcium concentration and was not inhibited by apamin. The amplitudes of the SK3 and Kᵥ currents varied from cell to cell and are summarized in FIG. 4.

Expression of GFP-tagged SK3-1B (SK3-1B-GFP) in PC12 cells abolished the native SK3 current without affecting the endogenous Kᵥ current (cf FIG. 3c with a). Untagged SK3-1B in a bicistronic vector containing GFP under translational control of an IRES element (SK3-1B-IRES-GFP) had a similar suppressive effect (FIG. 3d). To control for possible artifacts due to transfection and GFP overexpression, patch clamp experiments were performed on PC12 cells transfected with the GFP vector alone. SK currents in GFP transfected cells were sensitive to apamin and of comparable amplitude to untransfected cells (cf FIG. 3e with a). Patch-clamp experiments were also performed on PC12 cells transfected with a GFP tagged voltage-gated potassium channel, Kv1.7, only distantly related to SK channels, to ensure that dominant-negative suppression by SK3-1B was specific (FIG. 3f). In these cells, a large Kv1.7 current was seen, which was inward at −40 to 0 mV, and outward beyond 0 mV, consistent with the Nernst potential for potassium. Despite the presence of this substantial Kᵥ current, the amplitude of the apamin-sensitive SK currents (seen at potentials more negative than −40 mV) was indistinguishable from controls. The scatter plot in FIG. 4 summarizes the data and demonstrates that SK3-1B-GFP and SK3-1B-IRESGFP selectively suppress endogenous SK currents without affecting Kᵥ currents (means±SEM in FIG. 4). Together, these results indicate that SK3-1B suppresses native SK currents in PC12 cells specifically and in a dominant-negative fashion.

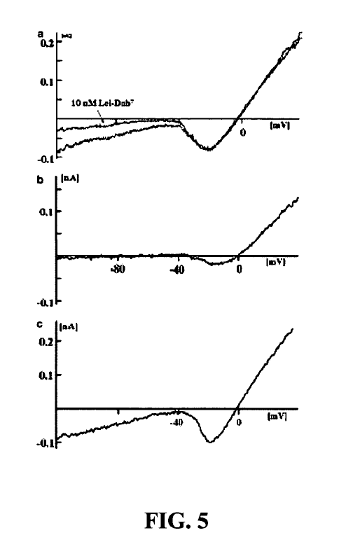

SK3-1B suppresses other SK channels in a dominant-inhibitory manner. The inventors next determined whether SK3-1B could suppress SK2 channels. A human Jurkat T lymphocyte line was chosen for these experiments because they express SK2 channels and no other SK channels, along with voltage-gated Kv1.3 channels that could serve as an internal control. Using the electrophysiological protocol described above for PC12 cells, SK2 and Kv1.3 currents were measured in Jurkat cells. The inward SK2 current, detected at potentials more negative than −40 mV, was blocked by Lei-Dab (FIG. 5a), a highly specific SK2 peptide inhibitor. Consistent with the Nernst potential for potassium, the Kv1.3 current was inward between −40 and 0 mV, and outward beyond 0 mV, and was unaffected by Lei-Dab (FIG. 5a). The native SK2 current was suppressed by SK3-1B, while the control Kv1.3 current was unaffected (FIG. 5b). This effect was specific since transfection of the GFP vector alone had no effect on either current (FIG. 5c). The ability of SK3-1B to suppress channels composed of SK3 and SK2 subunits suggests a potent form of negative dominant inhibition that may affect an entire sub-family of K⁺ channels, a channel family known to play a key role in regulating neuronal electrical firing frequency.

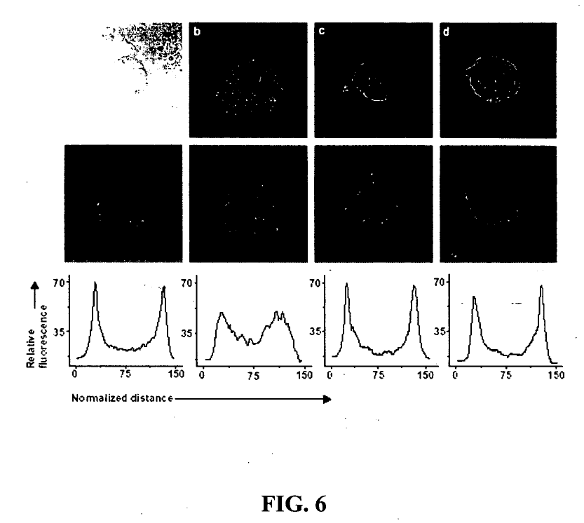

Dominant-negative suppression by SK3-1B sequesters native SK3. We performed immunolabeling and confocal microscopy experiments on PC12 cells to discern whether SK3-1B's dominant inhibitory effect was due to its ability to alter the subcellular localization of native SK3. A Nomarski image of a PC12 cell is shown in the upper panel of FIG. 6a. Endogenous SK3 protein was identified in this cell with an antibody that reacts only with the N-terminus of the full-length SK3 protein followed by a fluorescent (Alexa-594 conjugated) red secondary antibody. SK3 staining exhibited an annular pattern consistent with cell membrane expression (FIG. 6a, middle panel). Using Scion image software, we determined the pixel intensity of presumptive membrane and intracellular SK3 fluorescence, and the average intensity histogram is shown in the lower panel of FIG. 6a.

The subcellular localization of endogenous SK3 changed significantly following transfection of SK3-1B-GFP (cf FIG. 6b with a). In cells expressing SK3-1B-GFP (FIG. 6b, upper panel), native SK3 exhibited a red intracellular speckled pattern with diminished fluorescence intensity at the cell periphery (FIG. 6b, middle panel). The average intensity histogram of SK3 fluorescence (FIG. 6b, lower panel) was strikingly different from that of the native channel in untransfected cells. The sharp peaks evident in untransfected PC12 (lower panel, FIG. 6a) reflect the predominantly annular distribution of the native SK3 channel protein. In contrast, in SK3-1B, transfected cells, a greater proportion of the native SK3 protein is located intracellularly (lower panel, FIG. 6b). To control for nonspecific effects we used the same constructs used in the electrophysiology studies: GFP vector alone (FIG. 6c) or GFP-tagged Kv1.7 (FIG. 6d). These control constructs were expressed at a higher level (mean pixel intensity 170-180) than SK3-1B (mean pixel intensity: 108). Although GFP- and GFP-Kv1.7 transfected cells showed some degree of intracellular speckling (middle panels, FIG. 6c, d), the pattern was predominantly annular and the average intensity histogram data from multiple cells (lower panels, FIG. 6c, d) were similar to untransfected cells (lower panel, FIG. 6a). We estimated the ratio of presumptive membrane to intracellular SK3 fluorescence from the intensity histograms. The ratio in SK3-1B-GFP transfected PC12 cells (1.1±0.09; mean±SEM) was significantly different (p<0.001) from that in untransfected cells (1.72±0.08) or in cells transfected with GFP vector (1.98±0.09) or GFP-Kv1.7 (1.76±0.09). These results taken together with the electrophysiological analysis suggest that SK3-1B achieves dominant-negative inhibition of endogenous SK currents in PC12 and Jurkat cells by decreasing the abundance of functional channels in the plasma membrane, possibly by selectively coassembling with and sequestering native SK protein in intracellular compartments.

### Transgenic Animals

Transgenic animals comprise exogenous DNA incorporated into the animal's cells to effect a permanent or transient genetic change, preferably a permanent genetic change. Permanent genetic change is generally achieved by introduction of the DNA into the genome of the cell. Vectors for stable integration include plasmids, retroviruses and other animal viruses, YACs, and the like. Generally, transgenic animals are mammals, most typically mice.

The exogenous nucleic acid sequence may be present as an extrachromosomal element or stably integrated in all or a portion of the animal's cells, especially in germ cells. Unless otherwise indicated, a transgenic animal comprises stable changes to the germline sequence. During the initial construction of the animal, chimeric animals (chimeras) are generated, in which only a subset of cells have the altered genome. Chimeras may then be bred to generate offspring hemizygous for the transgene. Male and female hemizygotes are may then be bred to generate homozygous transgenic animals.

Typically, transgenic animals are generated using transgenes from a different species or transgenes with an altered nucleic acid sequence. For example, a human gene, such as the nucleic acid encoding SK3-1B, may be introduced as a transgene into the genome of a mouse. The introduced gene may be a wild-type gene, naturally occurring polymorphism, or a genetically manipulated sequence, for example having deletions, substitutions or insertions in the coding or non-coding regions. Where the introduced gene is a coding sequence, it is usually operably linked to a promoter, which may be constitutive or inducible, and other regulatory sequences required for expression in the host animal.

In general, the transgenic animals of the invention comprise transgenes that express SK3-1B, preferably human SK3-1B. Preferably, the introduced sequences provide for high expression of SK3-1B.

The transgenic animals of the invention can comprise other genetic alterations in addition to the presence of the SK3-1B-encoding sequences. For example, the host's genome may be altered to affect the function of endogenous genes (e.g., endogenous SK3-encoding genes), contain marker genes, or other genetic alterations consistent with the goals of the present invention.

### Nucleic Acid Compositions

Constructs for use in the present invention include any construct suitable for use in the generation of transgenic animals having the desired levels of expression of a desired SK3-1B-encoding sequence. Methods for isolating and cloning a desired sequence, as well as suitable constructs for expression of a selected sequence in a host animal, are well known in the art. In addition to the SK3-1B-encoding sequences, the construct may contain other sequences, such as a detectable marker.

The SK3-1B-encoding construct can contain a wild-type sequence encoding SK3-1B or mutant forms of SK3-1B, including nucleotide insertions, deletions, splice variants, and base substitutions, especially those associated with SK3-related pathologies in humans. The SK3-1B-encoding construct may include the open reading frame encoding specific polypeptides, introns, and adjacent 5′ and 3′ non-coding nucleotide sequences involved in the regulation of expression. The DNA sequences encoding SK3-1B may be cDNA or genomic DNA or a fragment thereof. The genes may be introduced into an appropriate vector for extrachromosomal maintenance or for integration into the host.

The nucleic acid compositions used in the subject invention may encode all or a part of SK3-1B as appropriate. Fragments may be obtained of the DNA sequence by chemically synthesizing oligonucleotides in accordance with conventional methods, by restriction enzyme digestion, by PCR amplification, and by other techniques known in the art.

Homologs of cloned SK3-1B may be identified by various methods known in the art. For example, nucleic acids having sequence similarity are detected by hybridization under low stringency conditions. Labeled nucleotide fragments can be used to identify homologous SK3-1B sequences as, for example, from other species.

The SK3-1B gene and exemplary derivatives thereof suitable for use in the production of the transgenic animals of the invention are described below.

### The SK3-1B Gene and its Derivatives Suitable For Use in the Present Invention

Transgenic animals of the present invention comprise a heterologous sequence encoding a desired SK3-1B gene, preferably a human SK3-1B gene. Preferably, the host animal produces high levels of human SK3-1B in neural cells. Preferably, the SK3-1B gene encodes a full-length human SK3-1B cDNA sequence. Alternatively, the SK3-1B gene can be an mutant, particularly an SK3-1B mutant associated with SK3-related pathology.

The host animals can be hemizygous or homozygous for the SK3-1B-encoding sequence. The SK3-1B gene can also be operably linked to a promoter to provide for a desired level of expression in the host animal and/or for tissue-specific expression. Preferably, SK3-1B gene expression is driven by a neuron-specific promoter. Expression of SK3-1B can be either constitutive or inducible.

### Methods of Making Transgenic Animals

Transgenic animals can be produced by any suitable method known in the art, such as manipulation of embryos, embryonic stem cells, etc. Transgenic animals may be made through homologous recombination, where the endogenous locus is altered. Alternatively, a nucleic acid construct is randomly integrated into the genome. Vectors for stable integration include plasmids, retroviruses and other animal viruses, YACs, and the like.

Specific methods of preparing the transgenic animals of the invention as described herein. However, numerous methods for preparing transgenic animals are now known and others will likely be developed. See, e.g., U.S. Pats. Nos. 6,252,131, 6,455,757, 6,028,245, and 5,766,879, all incorporated herein by reference. Any method that produces a transgenic animal expressing SK3-1B in neural cells is suitable for use in the practice of the present invention. The microinjection technique described is particularly useful for incorporating transgenes into the genome without the accompanying removal of other genes.

### Drug Screening Assays

The transgenic animals described herein may be used to identify compounds useful in the treatment of SK3-related pathologies. For example, transgenic animals of the present invention may be treated with various candidate compounds and the resulting effect, if any, on ataxia, intention tremors and hyperexcitability evaluated. Preferably, the compounds screened are suitable for use in humans.

Drug screening assays in general suitable for use with transgenic animals are known. See, for example, U.S. Pats. Nos. 6,028,245 and 6,455,757. The subject animals may be used by themselves, or in combination with control animals. Control animals may have, for example, a wild-type SK3 transgene that is not associated with SK3-related pathologies, or may be transgenic for a control construct that does not contain an SK3-1B-encoding sequence. The screen using the transgenic animals of the invention can employ any phenomena associated with SK3-related pathologies that can be readily assessed in an animal model.

### Therapeutic Agents

Once compounds have been identified in drug screening assays as eliminating or ameliorating the effects of SK3-related pathologies, these compounds can be used as therapeutic agents, provided they are biocompatible with the animals, preferably humans, to whom they are administered.

The therapeutic agents of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. Administration of the compounds can be administered in a variety of ways known in the art, as, for example, by oral, buccal, rectal, parenteral, intraperitoneal, intradermal, transdermal, intratracheal, etc., administration.

Depending upon the particular route of administration, a variety of pharmaceutically acceptable carriers, well known in the art can be used. These carriers include, but are not limited to, sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, 16th Edition, Mack, (1980)).

The concentration of therapeutically active compound in the formulation may vary from about 0.1-100 wt. %.

### EXAMPLES

### Example 1

### Generation and Maintenance of Tg Mouse Lines

Using transgenic technology we generated mice expressing SK3-1B under a neuron specific Thy 1 promoter. The clinical features of the 11 founders are listed in Table 1. Seven of the 11 founders exhibited mild to severe ataxia, intention tremor and hyperexcitable behavior. To ensure that the phenotype was Tg specific and not due to the site of integration disrupting normal genes, we have established two independent Tg lines. D2 (severe ataxia) and D11 (moderate ataxia) were selected as founders of these two lines (red in Table 1).

TABLE 1

| Founder | | | Ataxia | Hyper-excitable | |
|---|---|---|---|---|---|
| Name | Sex | Date of Birth | severity | behavior | Comments |
| A1 | Female | Apr. 04, 2002 | 0 | No | No obvious phenotype |
| A2 | Male | Apr. 04, 2002 | 3 | Present | Moderate ataxia |
| B1 | Male | Apr. 04, 2002 | 0 | No | No obvious phenotype |
| B2 | Female | Apr. 04, 2002 | 0 | No | No obvious phenotype |
| C3 | Female | Apr. 07, 2002 | 0 | No | No obvious phenotype |
| D2 | Male | Apr. 07, 2002 | 4 | Present | Severe ataxia, intention |
| | | | | | tremor |
| D9 | Female | Apr. 07, 2002 | 4 | Present | Severe ataxia, intention |
| | | | | | tremor |
| D11 | Female | Apr. 07, 2002 | 3 | Present | Moderate ataxia |
| E5 | Male | Apr. 07, 2002 | 0 | No | No obvious phenotype |
| F1 | Male | Apr. 07, 2002 | 2 | Present | Mild ataxia |
| F4 | Male | Apr. 07, 2002 | 1 | Present | Mild ataxia |
| F5 | Male | Apr. 07, 2002 | 5 | Present | Severe ataxia, intention |
| | | | | | tremor |

The scoring of the phenotype of the founders was done as follows. Ataxia: Based on relative performance on the hanging wire, balance on a beam and visible gait disturbance. 0: No phenotype, 1: Broad based gait. Walks on a beam of 4 mm diameter without difficulty. Hangs on wire for more than a minute. 2: Broad based gait, Slips <3 times on a beam but does not fall. Hangs on a wire for 30-60 s. 3: Broad based gait, slips <3 times on a beam but does not fall. Hangs on a wire 15-30 s 4: Broad based gait, slips multiple times on a beam and occasionally falls off the beam. Hangs on a wire <15 s. 5: Broad based gait. Invariably slips off a beam. Cannot hang on wire. Hyperexcitability: Based on observed motor activity and exploratory behavior.

### Example 2

### Breeding Hemizygous Tg Mice

As our parental strain we are currently using CB6F1 (hybrid of C57BL/6J X Balb/c) mice because of their known “hybrid vigor” and proclivity to produce large litters, an absolute necessity for all the Tg experiments of interest. We have already back-crossed D2 and D11 with normal CB6F1 mice to obtain “F1” litter. In the case of D11, an F1 male has been identified for further breeding to increase reproductive success. Since the phenotype penetrance is 100% we do not expect extreme variation in the behavior or electrophysiological changes in the Tg.

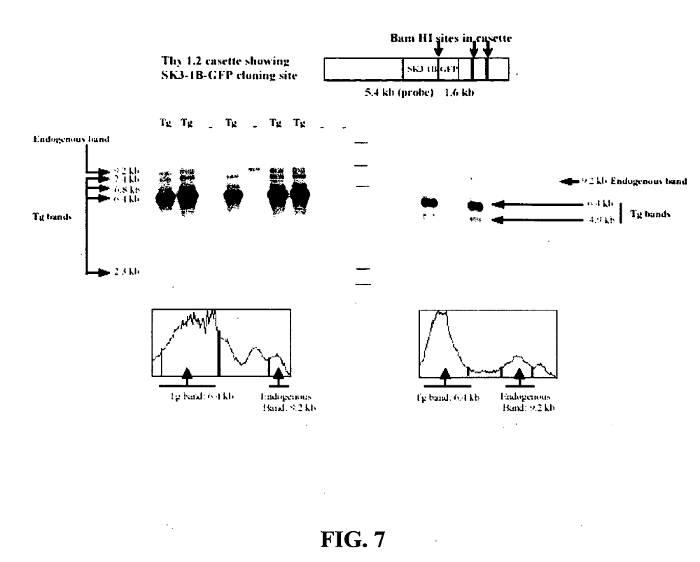

We have analyzed the F1 mice by Southern blot analysis (FIG. 7). All the lanes contain an endogenous band ˜9.2 kb. Tg mice contain in addition 4 Tg bands, the most prominent band being 6.4 kb. In the example shown, densitometry was performed on a D11 Tg-F1 and a D2 Tg F1 and the area under the curve for each band determined (FIG. 7, bottom panel). The Tg copy number was calculated by dividing the area under the principal Tg band by half the area under the endogenous band (since this band is derived from two alleles). We estimate that the D2 line has about eight Tg copies and D11 about 13 copies. We may be underestimating the intensity of the endogenous 9.1 kb band since it may not transfer as well as the smaller principal 6.4 kb Tg band, and therefore we may be over-estimating the Tg copy number in these mice. In any event, the Tg copy number in both lines is low (copy numbers >100 are considered high). Interestingly, D2 with severe ataxia has fewer Tg copies than D11 with moderate ataxia.

The D2 and D11 founder lines have been successfully established and the present inventors now have 30 Tg mice from each line. These mice exhibit the disease phenotype by about day 18. Additional mice may be bred as required.

### Example 3

### Analysis of Tg Expression and Pathology: Determining the Tg Product Expression Profile

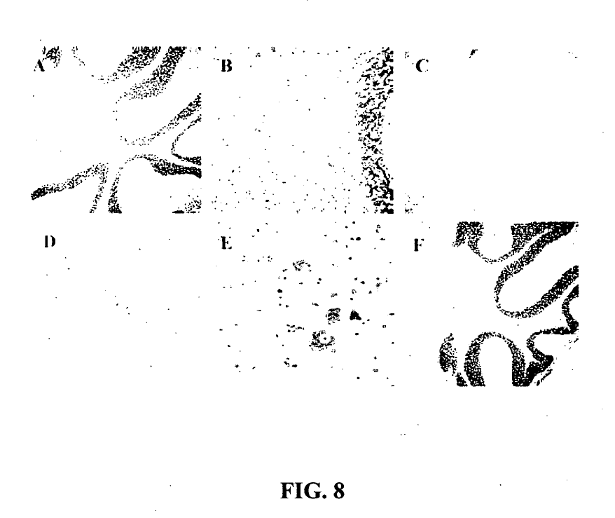

Half-the brain of each mouse was fixed in formalin and sent to American Histolabs (Gaithersburg, Md.) to prepare paraffin blocks and multiple sections for immunostaining and histological analysis. Brain sections from Tg mice and their non-Tg littermates were stained for SK3-1B-GFP expression using an immunoperoxidase sandwich method to correlate spatiotemporal expression of the Tg product with the disease phenotype. FIG. 8 shows a sample of Tg expression in different areas in the CNS including the DCN, neocortex and spinal cord. A summary of the distribution of the Tg in different brain regions in the two founder lines is shown in Table 2. The restricted distribution of the Tg product in the brain of both lines is consistent with the disease phenotype. For example, expression of the Tg in the DCN might contribute to the cerebellar ataxic phenotype since it comprises the core of the cerebellar circuitry, integrating excitatory and inhibitory information.

TABLE 2

| Founder line | CNS expression pattern |
|---|---|
| D2 | Deep cerebellar neurons (DCN), Brain stem |
| | nuclei (gigantocellular, pontine, red, |
| | cranial nerve), Thalamus, Subiculum, |
| | Layer V neocortex, spinal cord |
| D11 | DCN, CA1, Subiculum, Layer V neocortex. |
| | Brain stem nuclei (pontine, cranial nerve). |

### Example 4

### Does the Tg Produce Morphological Changes in the Brain?

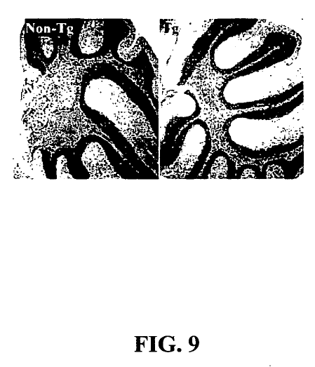

Histopathological studies of Tg and non-Tg F1 mice from each of the two Tg lines did not reveal obvious morphological changes in the brain as is evident in the representative hematoxylin and eosin-stained cerebellar sections shown in FIG. 9.

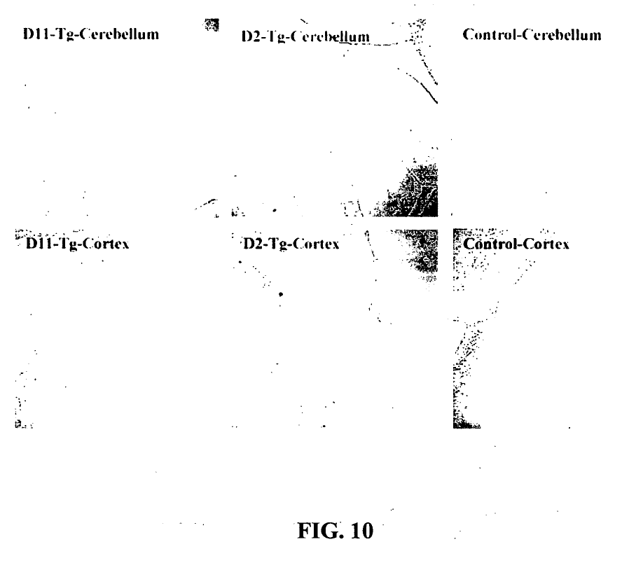

Reactive gliosis, an indirect marker for neurodegeneration was also not evident in sections stained for the glial marker glial fibrillary acidic protein (GFAP) with an anti-GFAP antibody in both lines (FIG. 10).

Sections stained with Fluoro-jade, a marker for degenerating neurons, also did not detect any difference between Tg and non-Tg F1s. Collectively, these results suggest that the disease phenotype in the SK3-1BGFP Tg mice is not associated with gross neurodegeneration.

### Example 5

### Behavior Tests

Behavioral tests are typically performed between the ages of 60-120 days. A two week handling period is necessary prior to the behavior task. Analysis was done at three time points—8 weeks, 12 weeks and 16 weeks to detect disease progression. The behavior tasks evaluated are shown in table 3.

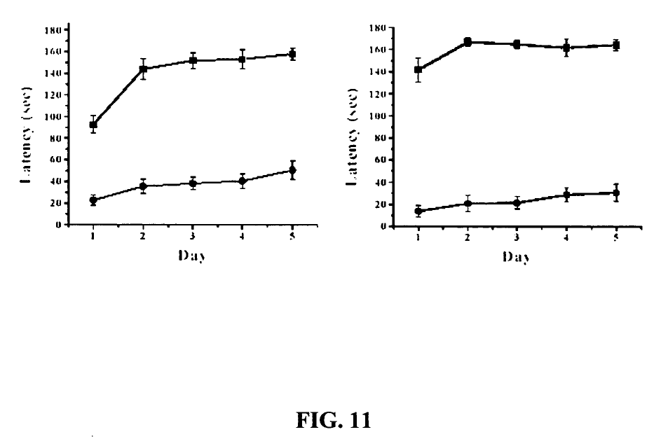

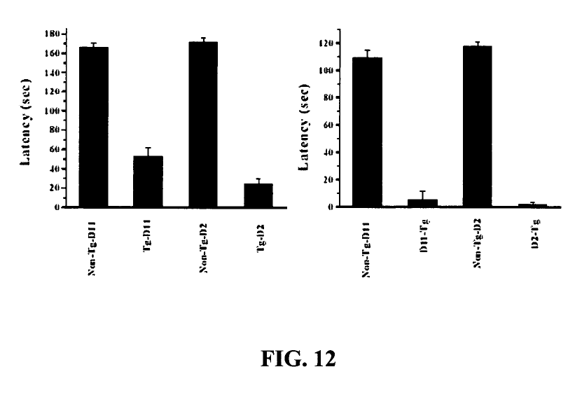

Behavioral data was statistically evaluated using analysis of variance (ANOVA) models containing one between-subjects variable (genotype) and one within-subjects variable (test sessions). Experiments on Tg and controls at 8 weeks are shown in FIGS. 11 and 12.

Rotorod experiments were performed daily for five consecutive days, six measurements being made each day for each mouse. Both Tg lines showed little motor learning with the D2 line again performing worse than the D11 line (FIG. 11).

Non-rotated rod and hanging wire experiments at 8 weeks showed that the Tg had impaired grip strength and equilibrium at rest (FIG. 12).

TABLE 3

| Test | Behavior evaluated |
|---|---|
| Nonrotated rod | Equilibrium |
| Rotating rotorod | Motor learning test (balance, grip, |
| | strength and somatosensory abilities) |
| Hanging wire | Grip strength |
| Footprint analysis | Motor coordination, gait |

Do Tg neurons exhibit spontaneous bursting activity? DCN neurons in rat cerebellum spontaneously fire at 35 spikes/sec, and SK blockade induces spontaneous bursts with intra-burst frequencies of 300 spikes/sec. Tg neurons appear to show irregular firing and short bursts at rest.

Those of skill will readily appreciate that dose levels can vary as a function of the specific therapeutic agents, the severity of the symptoms and the susceptibility of the subject to side effects. Preferred dosages for a given therapeutic agent are readily determinable by those of skill in the art by a variety of means. A preferred means is to measure the physiological potency of a given therapeutic agent.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

1. A transgenic mouse whose genome comprises a transgene encoding human small conductance calcium-activated potassium (SK) channel protein, splice variant B1 (“SK3-1B”), wherein the transgene is operably linked to a neuron-specific promoter, and wherein expression of the transgene results in ataxia.

2. The transgenic mouse of claim 1, wherein the expression of the transgene is limited to neurons.

3. The transgenic mouse of claim 1, wherein the mouse is fertile and transmits the SK3-1B transgene to its offspring.

4. The transgenic mouse of claim 1, wherein the SK3-1B transgene has been introduced into an ancestor of said mouse at an embryonic stage.

5. The transgenic mouse of claim 1, wherein the mouse is hemizygous for the human SK3-1B transgene.

6. The transgenic mouse of claim 1, wherein the mouse is homozygous for the human SK3-1B transgene.

7. The transgenic mouse of claim 1, wherein the mouse overexpresses SK3-1B in neurons relative to a control non-transgenic mouse.

8. The transgenic mouse of claim 1, wherein the promoter is a Thy1.2-SX promoter.

9. A transgenic mouse whose genome comprises a transgene encoding SK3-1B, wherein the transgene is operably linked to a neuron-specific promoter, and wherein expression of the transgene results in an intention tremor.

10. The transgenic mouse of claim 9, wherein the expression of the transgene is limited to neurons.

11. The transgenic mouse of claim 9, wherein the mouse is fertile and transmits the SK3-1B transgene to its offspring.

12. The transgenic mouse of claim 9, wherein the SK3-1B transgene has been introduced into an ancestor of said mouse at an embryonic stage.

13. The transgenic mouse of claim 9, wherein the mouse is hemizygous for the human SK3-1B transgene.

14. The transgenic mouse of claim 9, wherein the mouse is homozygous for the human SK3-1B transgene.

15. The transgenic mouse of claim 9, wherein the mouse overexpresses SK3-1B in neurons relative to a control non-transgenic mouse.

16. The transgenic mouse of claim 9, wherein the promoter is a Thy1.2-SX promoter.

17. A transgenic mouse whose genome comprises a transgene encoding SK3-1B, wherein the transgene is operably linked to a neuron-specific promoter, and wherein expression of the transgene results in hyperexcitable behavior.

18. The transgenic mouse of claim 17, wherein the expression of the SK3-1B transgene is limited to neurons.

19. The transgenic mouse of claim 17, wherein the mouse is fertile and transmits the SK3-1B transgene to its offspring.

20. The transgenic mouse of claim 17, wherein the SK3-1B transgene has been introduced into an ancestor of said mouse at an embryonic stage.

21. The transgenic mouse of claim 17, wherein the mouse is hemizygous for the human SK3-1B transgene.

22. The transgenic mouse of claim 17, wherein the mouse is homozygous for the human SK3-1B transgene.

23. The transgenic mouse of claim 17, wherein the mouse overexpresses SK3-1B in neurons relative to a control non-transgenic mouse.

24. The transgenic mouse of claim 17, wherein the promoter is a Thy1.2-SX promoter.

25. A method of screening biologically active agents that facilitate reduction of ataxia in vivo, the method comprising:
administering a candidate agent to a transgenic mouse according to claim 1, and
determining the effect of said agent upon the level of ataxia.

26. A method of screening biologically active agents that facilitate reduction of intention tremors in vivo, the method comprising:
administering a candidate agent to a transgenic mouse according to claim 9, and
determining the effect of said agent upon the level of intention tremors.

27. A method of screening biologically active agents that facilitate improvement in hyperexcitable behavior, the method comprising:
administering a candidate agent to a transgenic mouse according to claim 17, and
determining the effect of said agent upon hyperexcitable behavior.
